(19) United States
(12) Patent Application Publication
(10) Pub. No.: US 2010/0216847 A1
(43) Pub. Date: Aug. 26, 2010

(54) NORNICOTINE FOR THE TREATMENT OF PAIN

(75) Inventors: Joseph R. Holtman, JR., Lexington, KY (US); Peter A. Crooks, Nicholasville, KY (US); Linda Dwoskin, Lexington, KY (US); Elzbieta P. Wala, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: UNIVERSITY OF KENTUCKY RESEARCH FOUNDATION, LEXINGTON, KY (US)

(21) Appl. No.: 12/515,152

(22) PCT Filed: Nov. 16, 2007
(86) PCT No.: PCT/US07/24056

§ 371 Date: Feb. 24, 2010

Related U.S. Application Data
(60) Provisional application No. 60/859,488, filed on Nov. 17, 2006.

(51) Int. Cl. A61K 31/465; A61P 29/00

(52) U.S. Cl. 514/343

(57) ABSTRACT

This invention relates to the use of R(+)-, S(−)- or racemic nornicotine for the treatment of pain.

### FIELD OF THE INVENTION

This invention relates to the use of R(+), S(−)- or racemic nornicotine for the treatment of pain.

### BACKGROUND

Adequate management of pain is a critical health issue. Acute pain (e.g. postoperative pain) and chronic pain (e.g. arthritis, low back, and cancer pain) affects tens of millions of people annually in the United States alone. Each year about 30 million people visit a physician with a complaint of a painful condition. Over ten percent of these patients cite chronic pain as their main complaint. The financial loss worldwide due to pain has been estimated to exceed 100 billion dollars a year as a result of medical fees, lost productivity, litigation and the cost of drugs.

The primary group of drugs currently used to treat pain are the opioids, such as morphine, and non-steroidal anti-inflammatory (NSAID) agents, such as ibuprofen. However, these drug classes have significant side effects, limited efficacy, and/or other problems that limit their use for treating pain. In addition, while opioids and NSAIDs can be effective in treating nociceptive pain states (e.g. postsurgical pain and arthritis), they do not work well for chronic neuropathic pain which occurs as a result of injury to the peripheral or central nervous system. Neuropathic pain is characterized by central sensitization. Antidepressants (e.g. Elavil; Cymbalta) and anticonvulsants (e.g. Neurontin; Lyrica) have been used for neuropathic pain. However, their efficacy and use in different neuropathic pain states is also limited, and side effects are often an issue with their use. Thus, there is a need for more effective, less toxic drugs that can treat a broad range of pain.

One promising novel target for the development of drugs to treat pain is the nicotinic acteylcholine receptor (nAChRs). nAChRs are ion channels threaded through cell membranes. When activated, either by acetylcholine or other nicotinic receptor agonist drugs (e.g. nicotine), they allow selected ions to flow across the cell membrane. nAChRs are located in the central nervous system at sites known to be important in pain processing. A nAChR is composed of five polypeptide subunits. There are many nAChR subtypes made of different subunit combinations. Both acetylcholine and nicotine act at these receptors to alter electrochemical properties at a variety of synapses, which can in turn affect the release of several other neurotransmitters.

nAChRs play an important role in the control of pain, and thus, drugs acting at nicotinic receptors can be expected to have analgesic properties. This is true of nicotine. However, nicotine has marked side effects, which make its use as an analgesic undesirable. Nornicotine, the primary metabolite of nicotine, has been demonstrated to act at nicotinic receptors while having fewer side effects than nicotine. Thus, racemic nornicotine or one of its enantiomers, S(−)-nornicotine or R(+)-nornicotine, has potential as a drug for pain treatment.

### SUMMARY OF THE INVENTION

The present invention relates to the finding that S(−)-nornicotine, R(+)-nornicotine and/or racemic nornicotine can be used to alleviate pain. The present invention provides compositions and methods of using nornicotine or the treatment, alleviation and or prevention of pain.

The present invention provides a pharmaceutical composition comprising nornicotine and a pharmaceutically acceptable carrier.

The nornicotine may be R-(+)-nornicotine. The nornicotine may be S-(−)-nornicotine. The nornicotine may be the racemate.

The present invention also provides a method of treating, alleviating and/or preventing pain in a subject comprising administering to a subject in need thereof a pharmaceutically effective dose of nornicotine and/or its enantiomers.

The pain may be nociceptive. The pain ma be neuropathic. The pain may be acute. The pain may be chronic, nonmalignant and/or cancer pain.

### BRIEF DESCRIPTION OF THE FIGURES

FIG. 1 illustrates the analgesic effects of S(−)- and R(+)-nornicotine following intraperitoneal (IP) administration in a rodent model of nociceptive pain (tail-flick test). Time courses [A] and areas under the curves (AUC) [B] are presented (mean±SEM, n=6 rats).

FIG. 2 illustrates the analgesic effects of S(−)- and R(+)-nornicotine following intrathecal (IT) administration in a rodent model of nociceptive pain (tail-flick test). Time courses [A] and areas under the curves (AUC) [B] are presented (mean±SEM, n=5 rats).

FIG. 3 illustrates the dose-related analgesic effect of S(−)-nornicotine following intraperitoneal (IP) administration in a rodent model of nociceptive pain (paw withdrawal test). Time courses [A] and dose-response relationship [B] are presented (mean±SEM, n=8 rats).

FIG. 4 illustrates the analgesic effects of S(−)- and R(+)-nornicotine following intraperitoneal (IP) administration in a rodent model of chronic inflammatory pain associated with an acute (nociceptive) phase and a late (central sensitization) phase (formalin intra-plantar injection). Time courses [A] and areas under the curves (AUC) for early (acute) [B] and late (central sensitization) [C] phases in the formalin test are presented (mean±SEM, n=6 rats). The late phase is associated with central senstization and drugs active in this phase have been shown to be effective for neuropathic pain.

### DETAILED DESCRIPTION OF THE INVENTION

As described above, this invention provides compositions and formulations of nornicotine and/or its enantiomers, and methods of using of these agents, to treat pain. However, prior to describing this invention in further detail, the following terms will first be defined.

### DEFINITIONS

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless otherwise stated, the following terms used in the specification and claims have the meanings given below:

“Pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

“Treating” or “treatment” of a disease includes:

preventing the disease, i.e., causing the clinical symptoms of the disease not to develop in a mammal that may be exposed to or predisposed to the disease but does not yet experience or display symptoms of the disease

inhibiting the disease, i.e., arresting or reducing the development of the disease or its clinical symptoms, or

relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Pharmaceutically acceptable salt” refers to pharmaceutically acceptable salts of nornicotine which are salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Optional” or “optionally” means that the subsequently described event or circumstance may, but need not, occur, and that the description includes instances where the event or circumstance occurs and instances in which it does not.

The term “subject in need thereof” refers to any animal in need of relief from the symptoms of pain, or the same or similar symptoms caused by any other disease or condition. Preferably, the subject is a mammal. More preferably, the subject is human.

Pain can be broadly divided into two categories, nociceptive pain and neuropathic pain. Nociceptive pain occurs as a result of activation of peripheral nociceptors, free nerve endings, by noxious stimuli (e.g. heat, pressure, inflammatory mediators). Examples of nociceptive pain include postsurgical pain, inflammatory pain (e.g. arthritis) and low back pain. This pain is often described as being constant, dull and aching in nature. In contrast, neuropathic pain occurs as a result of damage to the peripheral or central nervous system. Examples of neuropathic pain include radiculopathy (e.g. disc impingement on a nerve, complex regional pain syndrome (CRPS I & II), and diabetic peripheral neuropathy] or central pain (e.g. pain from stroke, spinal cord injury, or multiple sclerosis). Patients typically describe neuropathic pain as “burning and tingling” in nature. It is characterized by allodynia (pain to a previously non-noxious stimulus) and hyperalgesia (increased painful response to a noxious stimulus).

In many patients, in particular those with chronic pain conditions of malignant (cancer-related pain) and non-malignant origin, pain is inadequately managed with currently available drugs. Current drugs or simple modifications of drugs belong to classes or medications which have been available for decades, including the opioids, nonsteroidal anti-inflammatory agents (NSAIDs) or adjuvants (antidepressants, anticonvulsants). Opioids are often used for the treatment of moderate to severe nociceptive pain. However, chronic neuropathic pain is much less responsive to opioids. Use of opioid analgesics is also associated with a broad range of significant side effects including cognitive impairment and respiratory depression. In addition, long-term opioid dosing results in the development of tolerance to the analgesic effect, drug abuse and dependence. The NSAIDs act by inhibition of the cyclo-oxygenase enzyme. However, the NSAIDs have limited efficacy when compared to the opioids. In addition, NSAIDs have significant side effects, including renal, gastrointestinal and cardiovascular problems. The discovery of the COX-2 selective agents (eg. Vioxx, Celebrex, Bextra) which have far less gastrointestinal toxicity was thought to be an advance in NSAID pharmacology. However, these agents still have low efficacy, and evidence is now available linking them to significant cardiovascular events including stroke and myocardial infarction following chronic use resulting in the removal of two of these agents, Vioxx and Bextra, from the market.

No suitable agent exists for the general treatment of neuropathic pain. Pregabalin (Lyrica), an anticonvulsant, has been approved for some specific neuropathic pain syndromes, including diabetic peripheral neuropathy, postherpetic neuralgia, but it still has limited efficacy. Duloxetine (Cymbalta), an antidepressant, has also been approved for diabetic peripheral neuropathy. N-methyl-D-aspartate (NMDA) receptor antagonists such as ketamine have been proposed for the treatment of neuropathic pain. However, their use alone is impractical, given their marked side effects, including sedation, psychosis and motor impairment. Thus, the limitations of the currently available therapies clearly demonstrate the need for a broad spectrum new class of efficacious and safe analgesic drugs for the treatment of nociceptive and neuropathic pain.

Given the need for more effective, less toxic, analgesic drugs, a great deal of emphasis has been placed on identifying novel molecular targets that could form the basis for new analgesics. One of the promising new targets is the neuronal nicotinic acetylcholine receptor (nAChR) [Decker M. W. et al., “Therapeutic potential of neuronal nicotinic acety receptor agonists as novel analgesics” Biochem Pharmacol (1999) 58:917-923; Decker M. W. et al., “Nicotinic acetylcholine receptor agonists: A potential new class of analgesics” Current Topics Med Chem (2004) 4:369-384; Flores C. M., “The promise and pitfalls of a nicotinic cholinergic approach to pain management” Pain (2000) 88:1-6; Williams M. et al., “Emerging molecular approaches to pain therapy” J Med Chem (1999) 42:1482-1500]. Nicotinic receptor agonists have been shown to have a broad spectrum of analgesic activity in several preclinical models of pain which simulate both nociceptive and neuropathic pain. This includes acute thermal pain models (tail flick, hot plate), inflammatory pain models (formalin or carrageenan injection into the paw) and nerve injury (neuropathic pain) models (spinal or sciatic nerve ligation) [Decker et. al., 2004]. Both anti-hyperalgesic and anti-allodynic effects were observed in the neuropathic pain models. Thus, nicotinic receptor agonists can be effective for both nociceptive and neuropathic pain.

Nicotinic receptor agonists, including nicotine and epibatidine (an alkaloid toxin from the skin of an Ecuadorian frog), have been studied as analgesics [Bannon A. W. et al., “Broad-spectrum, non-opioid analgesic activity by selective modulation of neuronal nicotinic acetylcholine receptors”, Science (1998a) 279:77-81; Curzon F. et al., “Differences between the antinociceptive effects of the cholinergic channel activators A-85380 and (±) epibatidine in rats” J Pharmacol Exp Ther (1998) 287:847-853; Damaj M. l. et al., “Tolerance to the antinociceptive effect of epibatidine after acute and chronic administration in mice” Eur J Pharmacol (1996a) 300:51-57; Damaj M. l. et al., “Characterization and modulation of acute tolerance to nicotine in mice” J Parmacol Exp Ther (1996b) 277:454-461; Damaj M. l. et al., “Antinociceptive responses to nicotinic acetylcholine receptor ligands after systemic and intrathecal administration in mice” J Pharmacol Exp Ther (1998) 284:1058-1065; Rao T. S. et al., “Evaluation of antinociceptive effects of neuronal nicotinic acetylcholine receptor ligands in the rat tail-flick assay” Neuropharmacology (1996) 35:393-405]. Several issues including limited duration of action, tolerance, narrow therapeutic index, lack of selectivity for neuronal nicotinic receptors, side effects (motor, cardiovascular, gastrointestinal, respiratory) have made these agents less than desirable analgesic drugs [Decker et. al., 2004]. Studies with ABT-594 [Bannon A. W. et al., “ABT-594 a novel, orally effective antinociceptive agent acting via neuronal nicotinic acetylcholine receptors: In vivo characterization”, J Pharmacol Exp Ther (1998b) 285:787-794; Boyce S. et al., “Analgesic and toxic effects of ABT 594 resemble epibatidine and nicotine in rats”, Pain (2000) 85:443-450; Decker M. W. et al., “The role of neuronal nicotinic acetylcholine receptors in antinociception: Effects of ABT-594” J Physiology (1998) 92:221-224; Donnelly-Roberts D. L. et al., “ABT-594[R0-5-(2-azetidinylmethoxy)-chloropyridine]: a novel, orally effective antinociceptive agent acting via neuronal nicotinic acetylcholine receptors: I. In vitro characterization” J Pharmacol Exp Ther (1998) 285:777-786], have begun to demonstrate more convincingly the plausibility of developing a nicotinic receptor agonist as an analgesic.

Activation of neuronal nicotinic receptors has also been shown to produce analgesia in humans [Flood P. et al., “Intranasal nicotine for postoperative pain treatment” Anesthesiology, (2004) 101:1417-1421]. Again, issues including those related to toxicity (cardiovascular, gastrointestinal, respiratory, motor), duration of action (short half-life), tolerance and abuse liability remain a concern [Flores 2000, for review].

### Nornicotine

Nornicotine is an active metabolite of nicotine. Nornicotine is preferred over nicotine, as nornicotine has better oral bioavailability, longer half life and a better side-effect profile than nicotine. Currently, nornicotine has been proposed for use as a tobacco use cessation agent [Ghosheh O. A. et al., “Residence times and half-lives of nicotine metabolites in rat brain after acute peripheral administration of [2′-¹⁴C]nicotine” Drug Metab Dispos (1999) 27:1448-1455; Ghosheh O. A. et al., “Accumulation of nicotine and its metabolites in rat brain after intermittent or continuous peripheral administration of [2′-¹⁴C]nicotine” Drug Metab Dispos (2001) 29:645-651; Dwoskin L. P. et al., “Acute and chronic effects of nornicotine on locomotor activity in rats: altered response to nicotine” Psychopharmacology (1999) 145:442-451; Stairs D. J. et al., “Enantiomeric effects of nornicotine on intravenous nicotine self-administration, dopamine metabolism and cardiovascular function in rats” J. Pharmacol Exp Ther (in press)].

Evidence suggests that the pharmacological profile of nornicotine resembles that of nicotine. However, nornicotine is less potent relative to nicotine in its dependence-producing properties [Bardo M. T. et al., “S(−)-nornicotine partially substitutes for R(÷) amphetamine in a drug discrimination paradigm in rats”, Pharmacol Biochem Behav, (1997) 1083-1087; Green T. A. et al., “Nornicotine pretreatment decreases intravenous nicotine self-administration” Psychopharmacology (2000) 152:289-294; Risner M. E. et al., “Effects of nicotine, cocaine, and some of their metabolites on schedule controlled responding by beagle dogs and squirrel monkeys” J Pharmacol Exp Ther (1985) 234:113-119; Risner M. E. et al., “Effects of stereoisomers of nicotine and nornicotine on schedule controlled responding and physiological parameters of dogs” Pharmacol Exp Ther (1988) 244:807-813]; behavioral sensitization and with respect to its cardiovascular effect Mattila M. “Pharmacological properties of some pyrrolidine N-substituted nornicotine derivatives” Ann Med Exp Biol Fenn (1963) 41:1-92; Stairs et al., in press].

Nornicotine is detectable in the urine from smokers and nicotine-treated laboratory animals. Metabolism of nicotine to nornicotine via N-demethylation is a minor pathway in the periphery [Cundy K. C. et al., “High performance liquid chromatographic method for the determination of N-methyl metabolites of nicotine” J Chromatogr Biomed Appl (1984) 306:291-301], while formation of nornicotine appears to be a major metabolic route in the central nervous system [Crooks P. A. et al. “Determination of nicotine metabolites in rat brain after peripheral radiolabeled nicotine administration: detection of nornicotine” Drug Metab Dispos (1995) 23:1175-1177; Crooks P. A. et al., “Contribution of CNS nicotine metabolites to the neuropharmacological effects of nicotine and tobacco smoking” Biochem Pharmacol (1997) 54:743-753; Crooks P. A. et al., “Metabolites of nicotine in rat brain after peripheral nicotine administration: cotinine, nornicotine and nornicotine” Drug Metab Dispos (1997) 25:47-54]. Nornicotine has a substantially longer plasma half-life compared to nicotine in humans (8 hours versus 1 hour) [Kyerematen G. A. et al., “Disposition of nicotine and eight metabolites in smokers and non-smokers: identification in smokers of two metabolites that are longer lived than cotinine” Clin Pharmacol Ther (1990) 48:641-651]; nornicotine resides about 3 times longer than nicotine (166 min vs. 52 min) in the rat's brain following peripheral administration of nicotine [Ghosheh et. al., 1999]. Furthermore, nornicotine accumulates in brain (about 4-fold compared to nicotine) following repeated nicotine dosing [Ghosheh et. al., 2001]. Nornicotine has good oral bioavailability, unlike nicotine, which is only 10% orally bioavailable.

Nornicotine appears to be less potent than nicotine with respect to its discriminative stimulus effects [Bardo et al., 1997]; reinforcement [Bardo M. T. et al., “Nornicotine is self-administered intravenously by rats”, Psychopharmacology, (1999) 146:290-296]; its effects on schedule controlled operant responding [Risner et al., 1985]; suppression of nicotine self-administration [Green et al., 2000] and behavioral sensitization [Dwoskin et. al., 1999]. Finally, blood pressure and autonomic side effects of nornicotine in cats and rats were less pronounced compared to nicotine [Mattila 1963; Stairs et al., in press]. The pharmacokinetic (accumulation in brain, long half-life, oral bioavailability) and diminished side effect profile make nornicotine and/or its enantiomers viable candidates to explore as analgesic agents for pain.

Initial studies suggest that nornicotine produces stereoselective effects on locomotor activity, schedule-controlled operant responding, abuse liability, and with respect to its autonomic side effects [Dwoskin et al., 1999; Risner et al., 1988; Stairs et al. in press]. This suggests that it may be possible to separate the desirable analgesis effect from the undesirable side effects of this nicotinic receptor agonist.

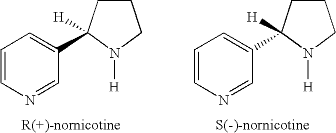

Nornicotine is present in S(−) and R(+) enantiomeric forms, as well as the racemate. The present invention contemplates the administration of R(+)-, S(−)-, and/or racemic nornicotine in order to achieve the treatment, alleviation and/or prevention of pain. Dose escalation studies show that S(−)-nornicotine may exhibit a more favorable potency and toxicity profile than the R(+)-nornicotine. Thus, S(−)-nornicotine may have advantages over R(+)-nornicotine for the treatment of pain.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

In general, the nornicotine compounds of the subject invention will be administered in a therapeutically effective amount by any of the accepted modes of administration for agents that serve similar utilities. The compounds can be administered in a variety of ways, including but not limited to: parenteral including subcutaneous (SC), intraperitoneal (IP), intravenous (IV), intraarterial, intramuscular (IM), transdermal, intradermal, intralesional, nasal, sublingual, vaginal, epidural, itrathecal (IT), intracerebroventricular (ICV), pulmonary (inhalation) and transmucosal, as well as oral (PO) and rectal routes. The compounds can be administered continuously by infusion or by the bolus injections. Preferably, the nornicotine compounds can be administered by the oral route. Such compositions are prepared in a manner well known in the pharmaceutical art and comprise at least one active compound.

The actual amount of the nornicotine will depend on a number of factors, such as the severity of the pain to be treated, the age and relative health of the subject, the potency of the used compound, the route and form of administration and other factors.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

In preparing the compositions of this invention, the active ingredient is usually mixed with an excipient, diluted by an excipient or enclosed within a carrier which can be in the form of a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, pessaries, sterile injectable solutions, and sterile packaged powders.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically or therapeutically effective amount. It will be understood, however, that the amount of the compound actually administered will be determined by a physician, in the light of the relevant circumstances, including the condition to be treated, the severity of the disease being treated, the chosen route of administration, the actual compound administered, the age, weight, and response of the individual patient, the severity of the patient's symptoms, and the like.

The dosage regimen for the composition of the present invention will, of course, vary depending upon known factors, such as the pharmacodynamic characteristic of the particular agent and its mode and route of administration; the species, age, sex, health, medical condition, and weight of the recipient; the nature and extent of the symptoms; the kind of concurrent treatment; the frequency of treatment; the route of administration; the renal and hepatic function of the patient, and the effect desired. An ordinary skilled physician or veterinarian can readily determine and prescribe the effective amount of the drug required to prevent, counter, or arrest the progress of the painful condition. Dosage forms (pharmaceutical compositions) suitable for administration may contain from about 1 milligram to about 1 gram of active ingredient per dosage unit. In these pharmaceutical compositions the active ingredient will preferable be present in an amount of about 0.5-95% by weight on the total weight of the composition. Advantageously, composition of the present invention may be administered in a single daily dose, or the total daily dosage may be administered in divided daily doses as needed. Typically, the clinician will administer the compound until a dosage is reached that achieves the desired effect.

In general, the nornicotine compounds of the invention will be administered in a therapeutically effective amount by any of the accepted modes of administration as previously stated.

In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the pain, the age, weight and general condition of the patient, and the like.

The compositions administered to a patient are in the form of pharmaceutical compositions described supra. These compositions may be sterilized by conventional sterilization techniques, or may be sterile filtered. When employed as pharmaceuticals, the compounds of the subject invention are usually administered in the form of pharmaceutical compositions. This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient or enclosed within a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, pessaries, sterile injectable solutions, and sterile packaged powders.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and/or flavoring agents. By way of example, for preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 1-500 mg of the active ingredient. The compositions of the invention can be formulated so as to provide immediate, sustained or delayed release of the active ingredient after administration to the patient by employing procedures known in the art.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The preferred parenteral form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Also included may be carrier molecules such as proteoglycans. Specific examples of such carrier molecules include, but are not limited to, glycosaminoglycans such as heparin sulfate, hyaluronic acid, keratan-sulfate, chondroitin 4-sulfate, chondroitin 6-sulfate, heparan sulfate and dermatin sulfate, perlecan, and pento polysulfate.

The amount of compound in the carrier solution is typically between about 1-500 mg. The dose administered will be determined by route of administration. Preferred routes of administration include parenteral or intravenous, epidural and intrathecal administration. A therapeutically effective dose is a dose effective to produce a significant decrease in pain.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. The compositions may be administered by the oral or nasal and pulmonary respiratory (e.g. inhalation) route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally, nasally including nasal spray and nasal drops or sublingually including sublingual spray and sublingual tablets, from devices which deliver the formulation in an appropriate manner.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches including transdermal carbon fiber needle patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid-soluble and amenable to transportation across the blood-brain barrier. Alternatively, the delivery of hydrophilic drugs may be enhanced by intra-arterial infusion of hypertonic solutions which can transiently open the blood-brain barrier.

The compounds of this invention can be administered in a sustained release form. Suitable examples of sustained-release preparations include semipermeable matrices of solid hydrophobic polymers containing the drug, which matrices are in the form of shaped articles, e.g., films, or microcapsules. Examples of sustained-release matrices include polyesters, hydrogels (e.g., poly(2-hydroxyethyl-methacrylate) as described by Langer et al., Biomed Mater Res. 15: 167-277 (1981) and Langer, Chem. Tech. 12: 98-105 (1982) or poly(vinyl alcohol)), polylactides (U.S. Pat. No. 3,773,919), copolymers of L-glutamic acid and gamma ethyl-L-glutamate [Sidman et al., Biopolymers 22: 547-556, 1983], non-degradable ethylene-vinyl acetate (Langer et al., supra), degradable lactic acid-glycolic acid copolymers such as the LUPRON DEPOT™ (i.e. injectable microspheres composed of lactic acid-glycolic acid copolymer and leuprolide acetate), and poly-D-(−)-3-hydroxybutyric acid (EP 133,988). The compounds of this invention can be administered in a sustained release form, for example a depot injection, implant preparation, or osmotic pump, which can be formulated in such a manner as to permit a sustained release of the active ingredient. Implants for sustained release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host. The implant is placed in proximity to the site of protein deposits (e.g., the site of formation of amyloid deposits associated with neurodegenerative disorders), so that the local concentration of active agent is increased at that site relative to the rest of the body.

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka et al., U.S. Pat. Nos. 4,235,871, 4,501,728 and 4,837,028 each of which is incorporated herein by reference.

The nornicotine can be further combined with other compounds or compositions used to treat, ameliorate or palliate pain. Dosage forms of the agents to be used in combination with the compounds and compositions disclosed herein would vary depending on the subject and drug combination being utilized.

The benefit of such combination therapies is that it may further lessen the class-specific and agent-specific side effects currently encountered with some of the drugs. Combinations of drugs that can lessen the quantity of a particular drug administered may reduce adverse side effects experienced by a patient.

The methods of the invention can be used to treat a patient that is affected with pain, or to prophylactically treat a patient at risk for severe pain, such as a patient about to undergo an operation. The dosage regimes necessary for prophylactic versus therapeutic treatment can vary, and will need to be designed for the specific use and disorder treated.

Those of skill will readily appreciate that dose levels can vary as a function of the specific agent, the severity of the symptoms and the susceptibility of the subject to side effects. Some of the specific agents are more potent than others. Preferred dosages for a given agent are readily determinable by those of skill in the art by a variety of means. A preferred means is to measure the physiological potency of a given agent.

In prophylactic applications, pharmaceutical compositions are chronically administered to a patient susceptible to, or otherwise at risk of, pain an amount sufficient to eliminate or reduce the risk or delay the onset of pain. Such an amount is defined to be a prophylactically effective dose.

The following examples are offered to illustrate this invention and are not to be construed in any way as limiting the scope of this invention.

### EXAMPLES

The purpose of these studies was to determine if S(−) and R(+) nornicotine have analgesic activity in rodent models of nociceptive (tail-flick test, thermal plantar) and inflammatory (intra-plantar formalin injection) pain. These rodent models of acute and chronic pain (as well as other rodent models of pain) have been used in our laboratory for testing the analgesic effects of opioids, benzodiazepines and NMDA-receptor antagonists [Holtman J. R. et al., “Modification of morphine analgesia and tolerance by flumazenil” Eur J Pharmacol (2003a) 470:149-156; Holtman J. R. et al., “Sex-related differences in the enhancement of morphine antinociception by NMDA receptor antagonists in rats” Pharmacol Biochem Behav (2003b); 76:285-293; Holtman J. R. et al., “(±)-Norketamine for treatment of neuropathic pain” AAPS (2004) W4083; Holtman J. R. et al., “Characterization of morphine-induced hyperalgesia in male and female rats” Pain (2005) 114:62-70; Johnson J. et al., “The antinociceptive effect of (−)-norketamine in an inflammatory pain model in rats” Exp. Biol. (2006) 168.5; Wala E. P. et al., “The effects of diazepam dependence and withdrawal on morphine-induced antinociception and changes in locomotion in male and female rats” Pharmacol Biochem Behav (2001) 69:475-484].

### Example 1

### Study of Analgesic Effects of S(−)- and R(+)-Nornicotine Following Intraperitoneal Administration (IP) in a Rodent Model of Nociceptive Pain; Tail-Flick Test

A study was performed to screen the analgesic activities of S(−) and R(+) enantiomers of nornicotine following administration by intraperitoneal route (IP) in a rodent model of nociceptive pain. The responses to acute thermal stimuli were determined using the tail-flick test [D′Amour F. E., Smith D. L. “A method for determining loss of pain sensation” J Pharmacol Exp Ther (1941), 72:74-79]. Tail-flick latency (TFL) was measured by recording the time from the onset of the heat stimulus to the tail to withdrawal of the tail from the heat source, using a standard tail-flick apparatus (EMDIE, Instrument Co., Roanoke, Va.). The sensitivity of the instrument was adjusted to provide an average baseline 2-3 seconds. Cut-off time of 10 s was used to avoid tail damage.

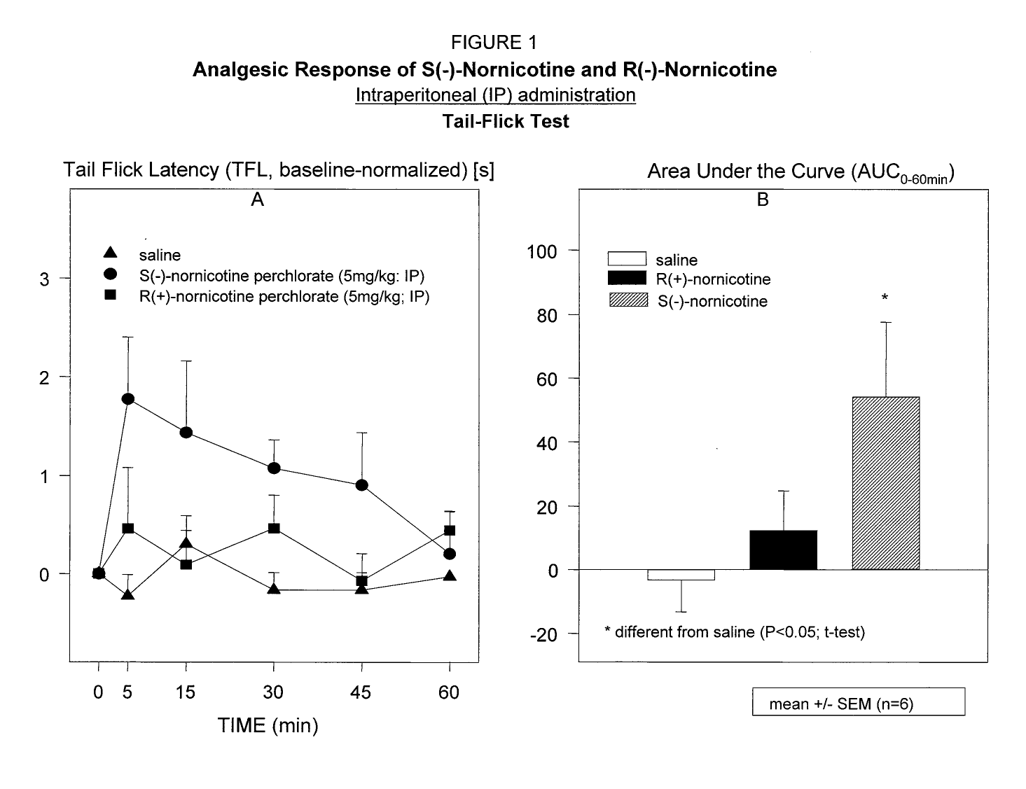

A single dose [5 mg/kg in perch salt form (perchlorate)] of each nornicotine enantiomer was administered (IP) in 6 male rats [Sprague-Dawley; about 90 days old, 350 g, (Harlan, Indianapolis, Ind.)]. Saline (vehicle, 1 ml/kg) served as control. TFL were determined prior to (twice, 15 min apart) and at 5, 15, 30, 45 and 60 min after i.p. administration. Data were presented as time courses of TFL (normalized for pre-injection baseline, s) [FIG. 1A] and areas under the curves (AUC₀₋₆₀ₘᵢₙ) [FIG. 1B]. These preliminary data demonstrated that the analgesic effect of S(−)-nornicotine (IP) was significantly greater as compared to saline (P<0.05; t-test). Analgesia of R(+)-nornicotine (IP) was less pronounced.

### Example 2

### Study of Analgesic Effects of S(−)- and R(+)-Nornicotine Following Intrathecal Administration (IT) in a Rodent Model of Nociceptive Pain; Tail-Flick Test

A study was performed to screen the analgesic activities of S(−) and R(+) enantiomers of nornicotine following administration by intrathecal route (IT) in a rodent model of nociceptive pain (tail-flick test).

In order to inject these drugs via the IT route chronic catheterization of the spinal subarachnoid space was performed in rats [Yaksh T., Rudy T. “Chronic catheterization of the spinal subarachnoid space” Physiol Behav (1976) 17:1031-1036 (with minor modification)]. Briefly, a 21 cm long P-10 polyethylene tubing (volume 10 mcl) which extended 8.5 cm beyond an incision in the atlanto-occipital membrane was secured to the scull with acrylic cement. The catheter rested in the vicinity of T-12 at the rostral face of the lumbar cord enlargement. The studies were initiated 1 week after implantation of the IT catheter.

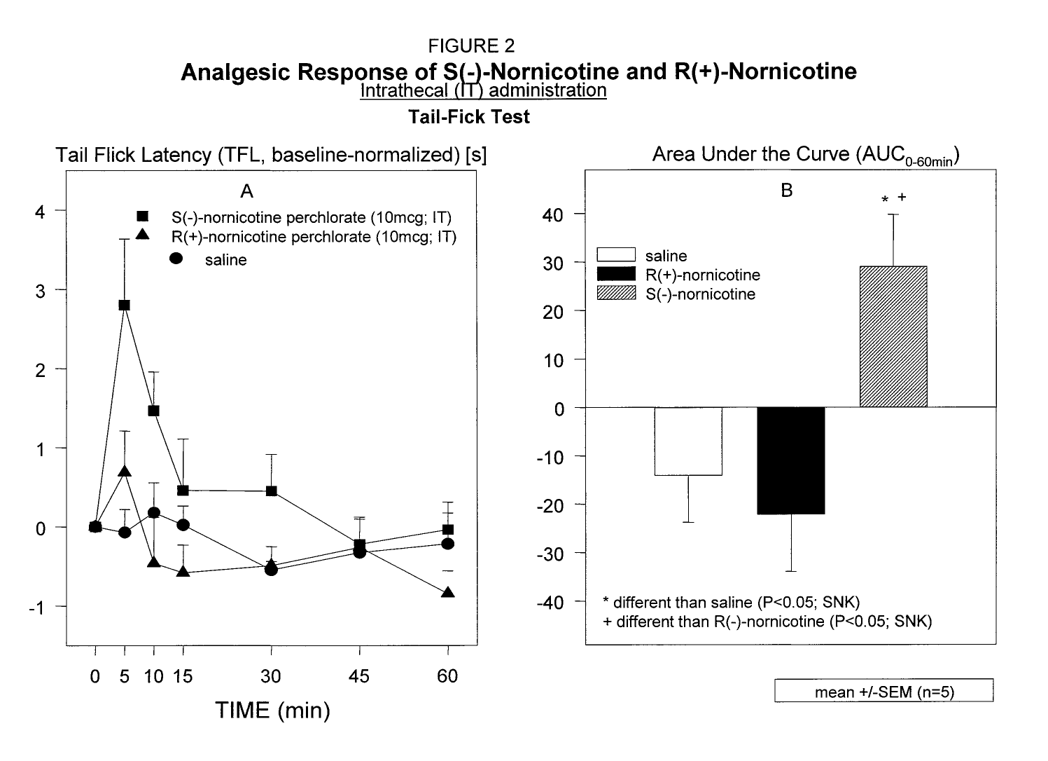

A single dose [10 mcg in perch salt form (perchlorate)] of each enantiomer was administered (IT) in 5 male rats. Saline (vehicle; 10 mcl) was used as control. The responses to acute thermal stimuli were determined using the tail-flick test (baseline 2-3 seconds, cut-off 10 seconds). TFL were measured prior to (twice, 15 min apart) and at 5, 10, 15, 30, 45 and 60 min after IT administration. Data were presented as time courses of TFL (normalized for pre-injection baseline, s) [FIG. 2A] and areas under the curves (AUC₀₋₆₀ₘᵢₙ) [FIG. 2B]. These preliminary data demonstrated that the analgesic effect of S(−)-nornicotine (IT) was significantly greater as compared to saline (P<0.05; post-hoc SNK). The analgesic effect of S(−)-nornicotine (IT) was significantly greater as compared to R(+)-nornicotine (IT) (P<0.05; post-hoc SNK).

### Example 3

### Study of Analgesic Effect of S(−)-Nornicotine Following Intraperitoneal Administration (IP) in a Rodent Model of Nociceptive Pain; Thermal Paw Withdrawal Test

To further characterize the analgesic properties of S(−)nornicotine, a dose-response relationship was determined. Responsiveness to acute thermal noxious stimuli was assessed by a thermal paw withdrawal test [Hargreaves K. et al., “A new sensitive method for measuring thermal nociception in cutaneous hyperalgesia” Pain (1988) 32:77-88]. This test, which uses a ramp heat stimulus on the plantar surface of the paw, appears to generate more consistent analgesic responses to nicotinic drugs as compared to tail flick test. Plantar Stimulator Analgesia Meter (11TC, Life Science, Woodland Hills, Calif.) was used. Briefly, the rat was placed in a clear plastic chamber and allowed to acclimate for 5 min. After the acclimation period, the radiant heat was positioned under the glass floor directly beneath the plantar hind paw. The heat source activated a timer that was controlled by a photocell. The hind paw withdrawal interrupted the photocell and automatically stopped the timer. Latency to paw withdrawal (PWL) was measured to the nearest 0.1 s. The calibration was such that the average PWL in untreated rats (baseline) was about 3-4 s (floor temperature equal to 30±0.1° C.). A maximum latency of 20 s was imposed if no response occurs within that time to prevent tissue damage.

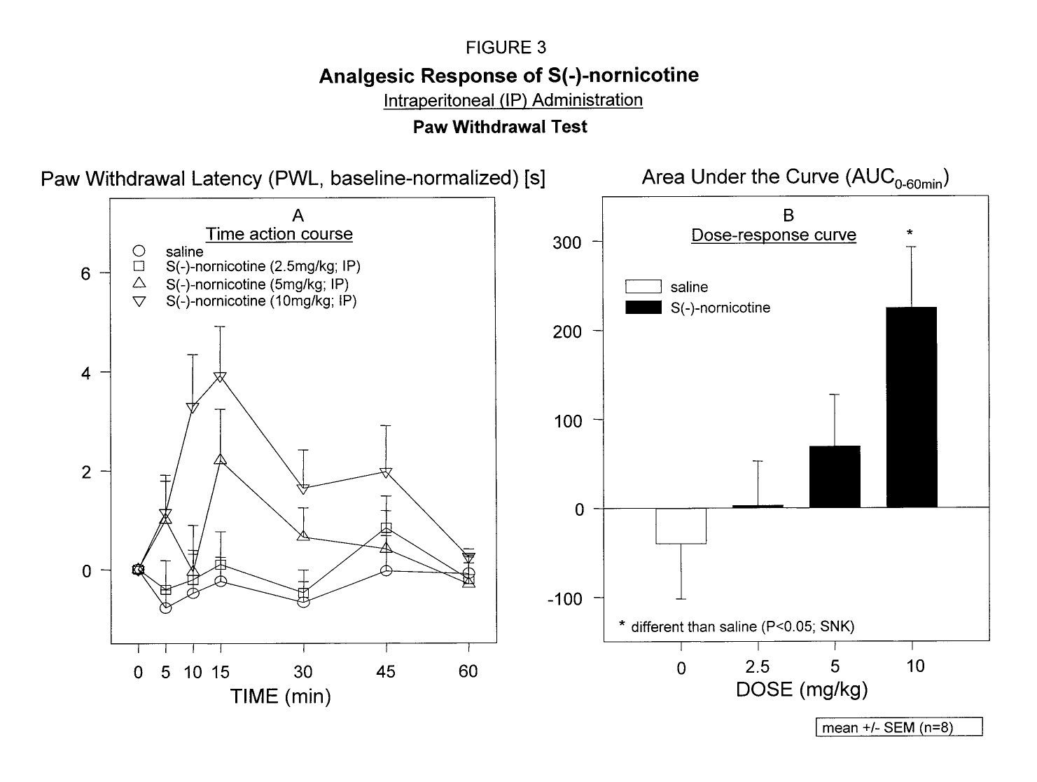

S(−)-Nornicotine was administered in three doses (2.5, 5 and 10 mg/kg in form of free base) via the IP route in 8 male rats. Saline (vehicle, 1 ml/kg) was also administered (control). Doses were balanced by the Latin-square design (4×4) and were administered at 48 h intervals. Responses (PWL, s) were measured prior to and at 5, 10, 15, 30, and 60 min after IP injection. For each rat and at each time point, response was normalized for baseline measured on the day of experiment. Data are presented as time courses of PWL (normalized for pre-injection baseline, s) [FIG. 3A] and dose-response relationship (AUC₀₋₆₀ₘᵢₙ vs. log dose) [FIG. 3B]. These data demonstrated that the analgesic effect of S(−)-nornicotine (IP) was significantly related to dose (P<0.05; 1-way ANOVA).

### Example 4

### Study of Analgesic Effect of S(−)- and R(+)-Nornicotine Following Intraperitoneal Administration (IP) in a Rodent Model of Inflammatory Pain; Formalin Test; Two Phase Test to Identify Drugs Active for Nociceptive Pain (Phase 1) and Neuropathic Pain (Phase 2)

A study was performed to screen the analgesic activities of S(−) and R(+) enantiomers of nornicotine following administration by intraperitoneal route (IP) in a rodent model of chronic inflammatory pain (formalin test). Intra-plantar injection of formalin produces a biphasic behavioral response consisting of flinching, lifting and licking behaviors in rodents. The first phase (acute, 0-20 min) results from direct stimulation of nociceptors (nociceptive pain) whereas the second phase (tonic, 20-60 min) involves central sensitization (a characteristic of neuropathic pain). Accordingly, the formalin test was used to determine the analgesic effects of S(−)- and R(+)-nornicotine [Wheeler-Aceto H., Covan A. “Standardization of the rat formalin test for the evaluation of analgesics” Psychopharmacology (1991) 104:35-44]. Formalin (50 mcl) was injected subcutaneously (SC) into the dorsal surface of the hind paw. The rat was placed in a Plexiglas cage and the intensity of pain (number of formalin-induced flinches) was rated in 5 min intervals for 60 min.

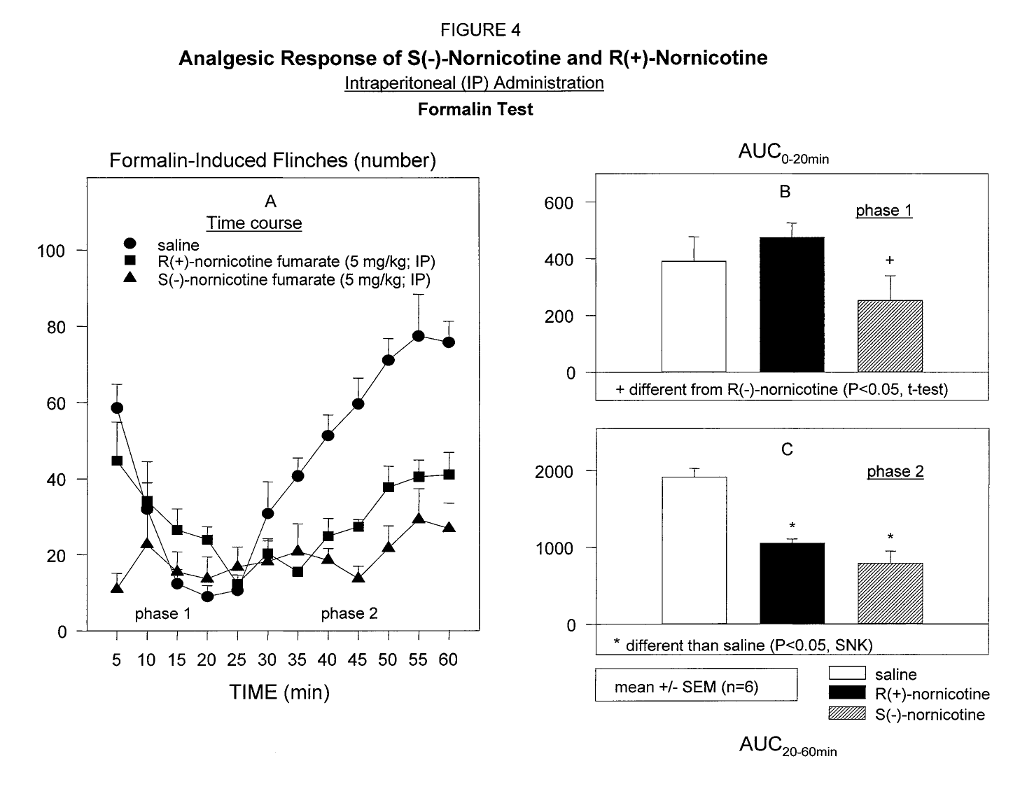

A single dose [5 mg/kg in salt form (fumarate)] of each nornicotine enantiomer and saline (control) was administered (IP) 15 min prior to formalin (SC) injection in 6 male rats. Data were presented as time courses of formalin-induced flinches (number) for S(−)-nornicotine, R(+)-nornicotine and saline [FIG. 4A] and areas under the curves (AUC) calculated for phase 1 (acute, 0-20 min) [FIG. 4B] and phase 2 (tonic, 20-60 min) [FIG. 4C]. These data demonstrated that both S(−)- and R(+)-nornicotine have analgesic efficacies in the chronic inflammatory pain model. This was evident as nornicotine attenuated formalin-induced flinching during the late (tonic) phase in the formalin test (significantly different from saline; P<0.05; post-hoc SNK). Neither enantiomer had significant effect in the early (acute) phase in the formalin test.

While the present invention has been described with reference to specific embodiments, this application is intended to cover those various changes and substitutions that may be made by those of ordinary skill in the art without departing from the spirit and scope of the appended claims.

What is claimed is:

1. A pharmaceutical composition comprising nornicotine and a pharmaceutically acceptable carrier.

2. The pharmaceutical composition of claim 1, wherein the nornicotine is R-(+)-nornicotine.

3. The pharmaceutical composition of claim 1, wherein the nornicotine is S-(−)-nornicotine.

4. The pharmaceutical composition of claim 1, wherein the nornicotine is the racemate.

5. A method of treating, alleviating and/or preventing pain in a subject comprising administering to a subject in need thereof a pharmaceutically effective dose of nornicotine and/or its enantiomers.

6. A method of treating pain of claim 5, wherein the pain is nociceptive.

7. A method of treating pain of claim 5, wherein the pain is neuropathic.

8. A method of treating pain of claim 5, wherein the pain is acute.

9. A method of treating pain of claim 5, wherein the pain is chronic, nonmalignant and/or cancer pain.
